(19) United States
(12) Patent Application Publication
(10) Pub. No.: US 2024/0382102 A1
(43) Pub. Date: Nov. 21, 2024

(54) MEDICAL IMAGE DIAGNOSTIC SYSTEM, OPERATION METHOD OF MEDICAL IMAGE DIAGNOSTIC SYSTEM, AND INFORMATION PROCESSING SYSTEM

(71) Applicant: FUJIFILM Healthcare Corporation, Chiba (JP)

(72) Inventors: Takeshi NAKAYAMA, Chiba (JP); Wataru YOKOHAMA, Chiba (JP)

(73) Assignee: FUJIFILM Healthcare Corporation, Chiba (JP)

(21) Appl. No.: 18/664,299

(22) Filed: May 15, 2024

(30) Foreign Application Priority Data
May 16, 2023 (JP) 2023-081129

(51) Int. Cl. A61B 5/055; A61B 5/00; A61B 5/1171; G16H 10/20

(52) U.S. Cl. CPC A61B 5/055; A61B 5/1171; A61B 5/7405; A61B 5/7435; G16H 10/20

(57) ABSTRACT

A medical image diagnostic system includes a first camera and/or a first microphone that detects, with at least one of a video or a voice, utterance-related information related to an utterance of a subject during an MRI examination, a second camera and/or a second microphone that detects, with at least one of a video or a voice, response information of the subject to a question to the subject before the examination is started, a projector, and a processor, in which the processor generates subject feature information related to voice generation of the subject based on the response information detected by the second camera and/or the second microphone, recognizes an utterance content of the subject based on the utterance-related information detected by the first camera and/or the first microphone and the subject feature information, and causes the projector to display the recognized utterance content.

### CROSS-REFERENCE TO RELATED APPLICATION

The present application claims priority under 35 U.S.C § 119 (a) to Japanese Patent Application No. 2023-081129 filed on May 16, 2023, which is hereby expressly incorporated by reference, in its entirety, into the present application.

### BACKGROUND OF THE INVENTION

### 1. Field of the Invention

The present invention relates to a medical image diagnostic system, an operation method of a medical image diagnostic system, and an information processing system, and particularly relates to a technique of reducing anxiety of a subject during an examination by an image diagnostic apparatus.

### 2. Description of the Related Art

An examination time for a subject using an image diagnostic apparatus, such as a magnetic resonance imaging apparatus (MRI apparatus) or an X-ray computed tomography (CT) apparatus, is relatively long, and is about 20 minutes to 30 minutes in a case of the MRI apparatus, and is about 5 minutes to 10 minutes in a simple examination and about 5 minutes to 20 minutes in a contrast examination in a case of the X-ray CT apparatus.

In addition, imaging using the MRI apparatus or the like is performed in a cylindrical imaging space called a “bore”, and since body movement can be an image artifact during the examination, it is necessary to suppress the body movement as much as possible including breathing. The examination in which the subject suppresses the body movement for a long time in a closed space called a “bore” is stressful for the subject.

During the examination, the subject may feel anxious and feel a poor physical condition in the closed space in which the body movement is not allowed. Even in a case in which the subject tries to convey the anxiety or the poor physical condition to a technician, there is a case in which the MRI apparatus has a loud examination sound and the examination sound does not reach the technician in an operation room.

JP2021-526048A discloses a technique of communicating between an operator of a magnetic resonance imaging system and a subject who is imaged by the magnetic resonance imaging system.

The magnetic resonance imaging system disclosed in JP2021-526048A acquires a moving image of a face region of the subject during acquisition of magnetic resonance imaging data, determines a voice activity state (an utterance state or a non-utterance state of the subject) from the acquired moving image, and displays “The utterance of the subject is detected” or displays an indicator for opening a communication channel on a dialog box opened on a user interface in a case in which the voice activity state indicates the utterance state, and in a case in which the communication channel is opened, the subject can talk with the operator using a subject microphone (the operator can listen a voice of the subject).

JP2021-526048A discloses that, by detecting a movement of a mouth (movement of a face) during utterance from the moving image of the face region of the subject, an event that is usually triggered by a patient pressing a balloon can be triggered, and thus the balloon can be omitted.

### SUMMARY OF THE INVENTION

JP2021-526048A discloses that the operator and the subject can have a conversation during a preparation stage of a first scan (silent period of a preparation phase) and between the subsequent scans, but does not disclose that the operator and the subject can have a conversation during the execution of a pulse sequence command. In particular, it is considered that the operator's voice cannot be heard due to the noise generated by the MRI apparatus during the execution of the pulse sequence command.

On the other hand, even during the execution of the pulse sequence command, in a case in which the voice activity state indicates the utterance state, the operator can hear the voice of the subject by performing noise cancellation or the like, but the subject cannot confirm whether or not the utterance content is transmitted to the operator.

That is, in the magnetic resonance imaging system disclosed in JP2021-526048A, although the anxiety or the poor physical condition of the subject during the examination can be transmitted to the operator by the voice, it is not possible to confirm whether or not the utterance content is reliably transmitted to the operator (whether or not the utterance content is connected to the outside), and it is not possible to resolve the anxiety of the subject being left alone in the examination room.

The present invention has been made in view of such circumstances, and an object of the present invention is to provide a medical image diagnostic system, an operation method of a medical image diagnostic system, and an information processing system which can reduce anxiety of a subject during an examination by an image diagnostic apparatus.

A first aspect of the present invention relates to a medical image diagnostic system comprising: an image diagnostic apparatus that acquires a medical image; a first detection device that detects, with at least one of a video or a voice, utterance-related information related to an utterance of a subject during an examination of the subject using the image diagnostic apparatus; a second detection device that detects, with at least one of a video or a voice, response information of the subject to a question to the subject before the examination of the subject using the image diagnostic apparatus is started; a first display device that displays information in an aspect visible to the subject during the examination using the image diagnostic apparatus; a processor; and a memory that stores a program to be executed by the processor, in which the processor generates subject feature information related to voice generation of the subject based on the response information detected by the second detection device, and recognizes an utterance content of the subject based on the utterance-related information detected by the first detection device and the subject feature information, to cause the first display device to display the recognized utterance content.

According to the invention according to the first aspect, the response information of the subject to the question to the subject is detected in advance by at least one of the video or the voice, and the subject feature information related to the voice generation of the subject is generated based on the response information. Moreover, in a case in which the utterance-related information related to the utterance of the subject is detected by at least one of the video or the voice during the examination of the subject using the image diagnostic apparatus, the utterance content of the subject is recognized based on the utterance-related information and the subject feature information, and the recognized utterance content is displayed on the first display device. As a result, the subject can visually recognize his or her own utterance content on the first display device, can confirm that the system understands his or her own utterance content, and can obtain a sense of security.

A second aspect of the present invention relates to the medical image diagnostic system according to the first aspect, in which the first detection device is preferably provided in a vicinity of the image diagnostic apparatus or the image diagnostic apparatus, and preferably includes at least one of a first camera that captures a video including at least a lip part of a face region of the subject during the examination using the image diagnostic apparatus, or a first microphone that detects a voice uttered by the subject.

A third aspect of the present invention relates to the medical image diagnostic system according to the second aspect, in which the utterance-related information is preferably a lip movement of the subject acquired from the video captured by the first camera. The analysis of the lip movement of the subject is performed to “read the lips” about the utterance content of the subject.

A fourth aspect of the present invention relates to the medical image diagnostic system according to any one of the first to third aspects, in which the second detection device is preferably provided at a position farther away from the image diagnostic apparatus than the first detection device, and preferably includes at least one of a second camera that captures a video including a face region of the subject, or a second microphone that detects a voice uttered by the subject.

A fifth aspect of the present invention relates to the medical image diagnostic system according to the fourth aspect, in which the response information is preferably a lip movement of the subject acquired from the video captured by the second camera. This is to acquire the feature (lip movement of the subject) during the utterance of the subject in advance.

A sixth aspect of the present invention relates to the medical image diagnostic system according to any one of the first to fifth aspects, preferably further comprising: a question apparatus that asks a question to the subject, in which the question apparatus asks a question of a predetermined format with at least one of a voice or characters on a monitor screen. It should be noted that the operator or the like who operates the image diagnostic apparatus is not hindered from asking a question to the subject.

A seventh aspect of the present invention relates to the medical image diagnostic system according to the sixth aspect, in which the question of the predetermined format preferably includes a question content for inducing an utterance having a possibility of being uttered by the subject during the examination, as an answer, or causing the subject to read aloud the utterance. This is to improve the recognition accuracy in a case of automatically recognizing the utterance content of the subject.

An eighth aspect of the present invention relates to the medical image diagnostic system according to any one of the first to seventh aspects, in which the processor preferably trains a first machine learning model dedicated to the subject based on the response information detected by the second detection device, and inputs the utterance-related information detected by the first detection device to the trained first machine learning model, to acquire the utterance content recognized by the first machine learning model. By using the first machine learning model customized for each subject, the recognition accuracy of the utterance content of the subject is improved.

A ninth aspect of the present invention relates to the medical image diagnostic system according to the eighth aspect, in which the subject feature information preferably includes parameters optimized in a process of training the first machine learning model based on response information of the subject.

A tenth aspect of the present invention relates to the medical image diagnostic system according to the eighth or ninth aspect, in which a second machine learning model that has been trained through machine learning in advance based on a training data set consisting of utterance-related information related to utterances of a plurality of people is preferably provided, and the processor preferably inputs the utterance-related information detected by the second detection device to the second machine learning model, to acquire the utterance content recognized by the second machine learning model in a case in which the utterance content recognized by the first machine learning model is not a meaningful content or a certainty degree of the utterance content is less than a threshold value. This is because there is a case in which the number of questions to the subject (response information of the subject to the question to the subject) is small and the first machine learning model is not sufficiently trained, or in a case in which a case of using the second machine learning model that has been trained using the training data set consisting of the utterance-related information related to the utterance of a large number of subjects has a higher recognition accuracy than a case of using the first machine learning model.

An eleventh aspect of the present invention relates to the medical image diagnostic system according to any one of the first to tenth aspects, preferably further comprising: a notification device that notifies an operator in an operation room of the image diagnostic apparatus of the utterance content, in which the processor outputs the utterance content to the notification device. As a result, the operator can also confirm the utterance content of the subject.

A twelfth aspect of the present invention relates to the medical image diagnostic system according to the eleventh aspect, in which the notification device is preferably at least one of a second display device that displays characters indicating the utterance content or a speaker that generates a voice indicating the utterance content.

A thirteenth aspect of the present invention relates to the medical image diagnostic system according to the third aspect, in which the processor preferably determines whether or not the lip movement of the subject hinders the examination of the subject using the image diagnostic apparatus in a case in which next utterance-related information is not detected for a certain time or longer after the first detection device detects the utterance-related information, and causes the first display device to display characters prompting the subject to make an utterance in a case in which it is determined that the lip movement of the subject does not hinder the examination of the subject using the image diagnostic apparatus. In a case in which the examination part is a part other than the head, it can be determined that the body movement (movement of the head) due to the utterance of the subject does not hinder the examination of the part other than the head. In this case, the characters prompting the subject to make the utterance are displayed on the first display device, so that communication between the subject and the operator can be performed, and the anxiety of the subject can be resolved.

A fourteenth aspect of the present invention relates to the medical image diagnostic system according to any one of the first to thirteenth aspects, preferably further comprising: a vital information measurement device that measures vital information of the subject during the examination of the subject using the image diagnostic apparatus, in which the processor determines whether or not a reply to the utterance content is necessary, based on the utterance content and the measured vital information, and creates a reply sentence corresponding to the utterance content to cause the first display device to display the reply sentence in a case in which it is determined that the reply is necessary. By acquiring the vital information of the subject in addition to the utterance content of the subject, it is possible to more appropriately determine the anxiety, the physical condition, and the like of the subject, and it is possible to provide necessary information to the subject.

A fifteenth aspect of the present invention relates to the medical image diagnostic system according to the fourteenth aspect, in which the vital information measurement device preferably measures one or more of a heart rate, a blood pressure, a respiratory rate, a body temperature, an electrocardiogram, or a blood oxygen saturation concentration of the subject.

A sixteenth aspect of the present invention relates to the medical image diagnostic system according to any one of the first to fifteenth aspects, in which the first display device is preferably a projector that performs projection onto a screen visible to the subject, a head-up display, a head-mounted display, a liquid crystal display, or an organic EL display.

A seventeenth aspect of the present invention relates to the medical image diagnostic system according to any one of the first to sixteenth aspects, in which the image diagnostic apparatus preferably includes a magnetic resonance imaging apparatus, an X-ray CT apparatus, a PET apparatus, a radiation therapy apparatus, or a particle beam therapy apparatus. The present invention is suitable for the image diagnostic apparatus in which the examination time and the treatment time are relatively long.

An eighteenth aspect of the present invention relates to an operation method of a medical image diagnostic system including a first detection device that detects, with at least one of a video or a voice, utterance-related information related to an utterance of a subject during an examination of the subject using an image diagnostic apparatus, a second detection device that detects, with at least one of a video or a voice, response information of the subject to a question to the subject before the examination of the subject using the image diagnostic apparatus is started, a first display device that displays information in an aspect visible to the subject during the examination using the image diagnostic apparatus, a processor, and a memory that stores a program to be executed by the processor, the operation method comprising: a step of generating, via the processor, subject feature information related to voice generation of the subject based on the response information detected by the second detection device; a step of recognizing, via the processor, an utterance content of the subject based on the utterance-related information detected by the first detection device and the subject feature information; and a step of causing, via the processor, the first display device to display the recognized utterance content.

A nineteenth aspect of the present invention relates to the operation method of a medical image diagnostic system according to the eighteenth aspect, preferably further comprising: a step of training, via the processor, a first machine learning model dedicated to the subject based on the response information detected by the second detection device; and a step of inputting, via the processor, the utterance-related information detected by the first detection device to the trained first machine learning model, to acquire the utterance content recognized by the first machine learning model.

A twentieth aspect relates to an information processing system comprising: a first detection device that detects, with at least one of a video or a voice, utterance-related information related to an utterance of a subject during an examination of the subject using an image diagnostic apparatus; a second detection device that detects, with at least one of a video or a voice, response information of the subject to a question to the subject before the examination of the subject using the image diagnostic apparatus is started; a first display device that displays information in an aspect visible to the subject during the examination using the image diagnostic apparatus; a processor; and a memory that stores a program to be executed by the processor, in which the processor generates subject feature information related to voice generation of the subject based on the response information detected by the second detection device, and recognizes an utterance content of the subject based on the utterance-related information detected by the first detection device and the subject feature information, to cause the first display device to display the recognized utterance content.

According to the present invention, the subject during the examination using the image diagnostic apparatus can confirm that his or her utterance content is transmitted to an external operator or the like, and can obtain a sense of security.

### BRIEF DESCRIPTION OF THE DRAWINGS

FIG. 1 is a view showing an outline of a layout of an image diagnosis facility to which a medical image diagnostic system according to an embodiment of the present invention is applied.

FIG. 2 is a perspective view showing an example of the image diagnostic apparatus.

FIG. 3 is a schematic cross-sectional view of an MRI apparatus shown in FIG. 2.

FIGS. 4A and 4B are views showing switching of a direction of a body of a subject with respect to the MRI apparatus.

FIG. 5 is a view showing a state inside an operation room.

FIG. 6 is a view showing an example of equipment installed in an interview room.

FIG. 7 is a configuration diagram of the medical image diagnostic system according to the embodiment of the present invention.

FIG. 8 is a block diagram showing an embodiment of hardware of a medical image diagnostic apparatus, which is a main device constituting the medical image diagnostic system.

FIG. 9 is a conceptual diagram showing an embodiment of an utterance recognition unit.

FIG. 10 is a diagram showing another embodiment of a first display device that displays information in an aspect visible to the subject during an examination using the MRI apparatus.

FIG. 11 is a flowchart showing an embodiment of an operation method of the medical image diagnostic system that is operated in a case in which the subject is in the interview room.

FIG. 12 is a flowchart showing a first embodiment of the operation method of the medical image diagnostic system during an MRI examination.

FIG. 13 is a flowchart showing a second embodiment of the operation method of the medical image diagnostic system during the MRI examination.

FIG. 14 is a flowchart showing a third embodiment of the operation method of the medical image diagnostic system during the MRI examination.

### DESCRIPTION OF THE PREFERRED EMBODIMENTS

Hereinafter, preferred embodiments of a medical image diagnostic system, an operation method of a medical image diagnostic system, and an information processing system according to an embodiment of the present invention will be described with reference to the accompanying drawings.

### Outline of Medical Image Diagnostic System

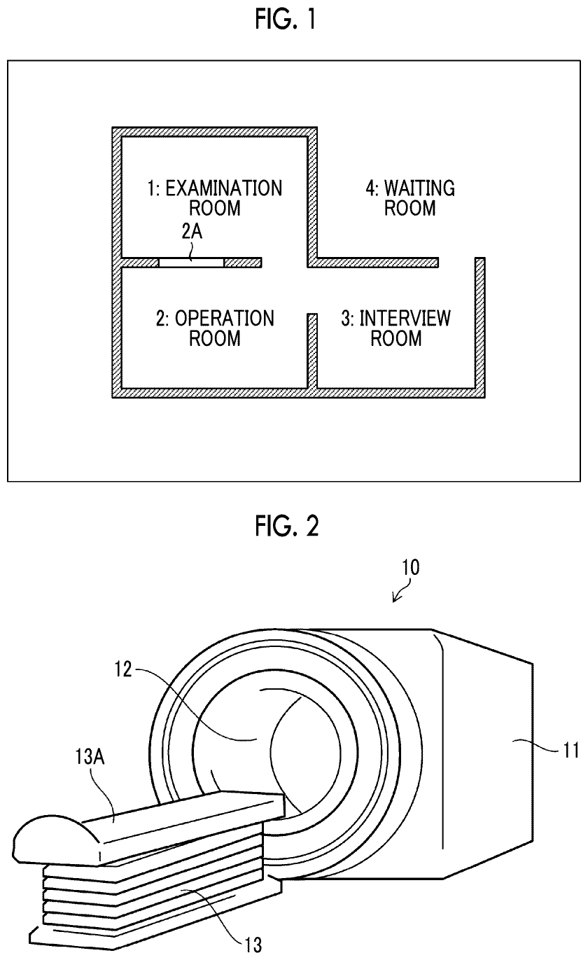

FIG. 1 is a view showing an outline of a layout of an image diagnosis facility to which the medical image diagnostic system according to the embodiment of the present invention is applied.

The image diagnosis facility shown in FIG. 1 is a part of a hospital or a health examination facility, and includes an examination room 1 in which an image diagnostic apparatus is installed, an operation room 2 in which an operator operates the image diagnostic apparatus, an interview room 3 in which an interview with a subject and the like is performed, and a waiting room 4 in which an attendant of the subject, such as a family, waits.

FIG. 2 is a perspective view showing an example of the image diagnostic apparatus.

An image diagnostic apparatus shown in FIG. 2 is a magnetic resonance imaging apparatus (MRI apparatus) 10, and comprises a gantry 11 and a bed 13 comprising a top plate 13A disposed on a front side of a bore 12 that is a cylindrical imaging space provided in the gantry 11.

The image diagnostic apparatus according to the present example is the MRI apparatus 10, but the present invention is not limited thereto, and includes an X-ray CT apparatus, a positron emission tomography (PET) apparatus, a radiation therapy apparatus, or a particle beam therapy apparatus. The present invention is suitable for an apparatus in which the examination time and the treatment time are relatively long.

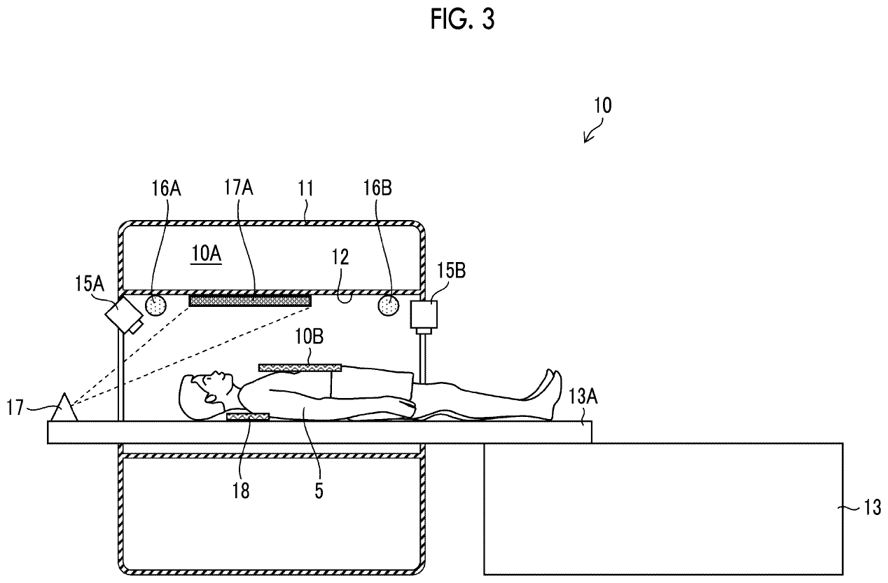

FIG. 3 is a schematic cross-sectional view of the MRI apparatus shown in FIG. 2.

In FIG. 3, an MRI magnet 10A and various coils are provided on the gantry 11, and a subject 5 lying face up on the top plate 13A is moved so that the examination part of the subject 5 is located at the center of the static magnetic field in the bore 12 by moving the top plate 13A to the bore 12. A radio frequency (RF) coil 10B for receiving free induction decay (FID) from a human body is disposed at the examination part of the subject 5. In the present example, the abdomen of the subject 5 is the examination part, and the RF coil 10B is disposed on the abdomen.

A first camera 15 (15A and 15B) and a first microphone 16 (16A and 16B), which are first detection devices, are disposed at the upper portions of both ends of the bore 12. It should be noted that, in FIG. 3, the first camera 15A and the first microphone 16A are disposed at the upper portion of the left end of the bore 12, and the first camera 15B and the first microphone 16B are disposed at the upper portion of the right end of the bore 12.

Each of the two first cameras 15A and 15B images the subject 5 and outputs a moving image (video), and at least one of the first camera 15A or the first camera 15B images a video including a face region of the subject 5.

Further, each of the two first microphones 16A and 16B detects a voice uttered by the subject 5. It is preferable that the first microphones 16A and 16B are non-magnetic microphones using a piezoelectric ceramic that is a non-magnetic material in a sound receiving element portion.

A projector 17 is provided at one end (in FIG. 3, the left end) of the top plate 13A, and the projector 17 projects the video onto a screen 17A that is provided in a ceiling portion in the bore 12. The projector 17 functions as a display device (first display device) that displays information in an aspect visible to the subject 5 during the examination using the MRI apparatus 10, and the subject 5 can visually recognize the video projected onto the screen 17A during the examination. It should be noted that the ceiling portion in the bore 12 may be used as a screen without providing the screen 17A.

In FIG. 3, 18 indicates a vital information measurement device that measures one or more of a heart rate, a blood pressure, a respiratory rate, a body temperature, an electrocardiogram, or a blood oxygen saturation concentration of the subject 5. The vital information measurement device 18 is not limited to being disposed on the back portion of the subject 5.

Specific examples of the vital information measurement device 18 include an electrocardiogram (ECG) sensor that measures the electrocardiogram and the heartbeat, an SpO2 sensor that measures the blood oxygen saturation concentration, a bellows that detects the respiratory rate, an optical camera that can detect a facial expression, the body temperature, and the like of the subject 5, an infrared camera, a thermography that measures the body temperature, a device that measures the vital information based on a nuclear magnetic resonance (NMR) signal (a signal itself acquired in an MRI examination), and an MRI image (an image obtained by imaging the NMR signal), other sensors (such as a pressure sensor, an optical sensor, and an ultrasound sensor), and a combination thereof.

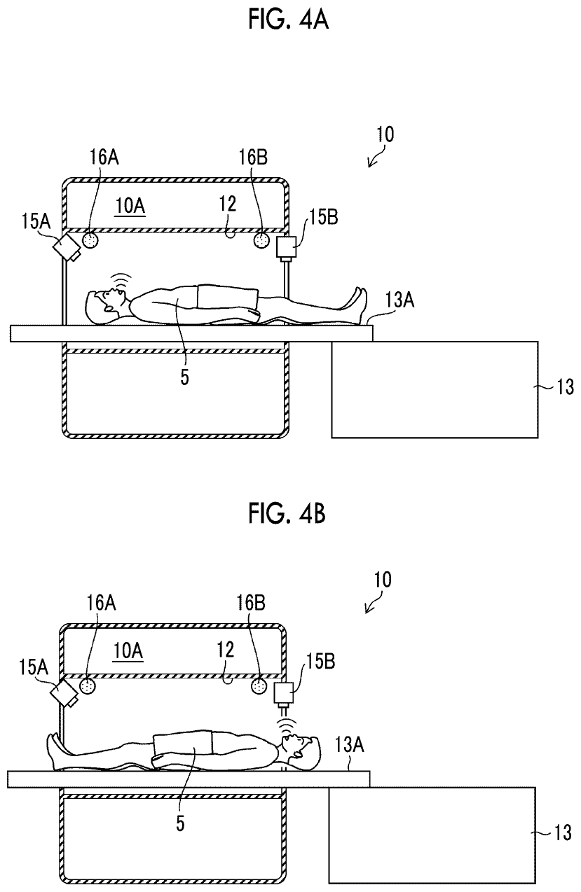

FIGS. 4A and 4B are views showing switching of a direction of a body of the subject with respect to the MRI apparatus.

FIG. 4A shows a case in which the subject 5 enters the bore 12 from the head (head first), and FIG. 4B shows a case in which the subject 5 enters the bore 12 from the foot (foot first).

The head first or the foot first is selected depending on the examination part of the subject 5. In the present example, in order to perform the imaging of the face region of the subject 5 and the detection of the voice regardless of whether the head first or the foot first is selected, the first cameras 15A and 15B and the first microphones 16A and 16B are disposed at both end portions of the bore 12, respectively.

It should be noted that the first camera 15 (15A and 15B) and the first microphone 16 (16A and 16B) are two in the present example, but are not limited thereto, and may be one or three or more. In addition, the first camera 15 and the first microphone 16 are not limited to being disposed in the MRI apparatus 10, and may be disposed in the vicinity of the MRI apparatus 10.

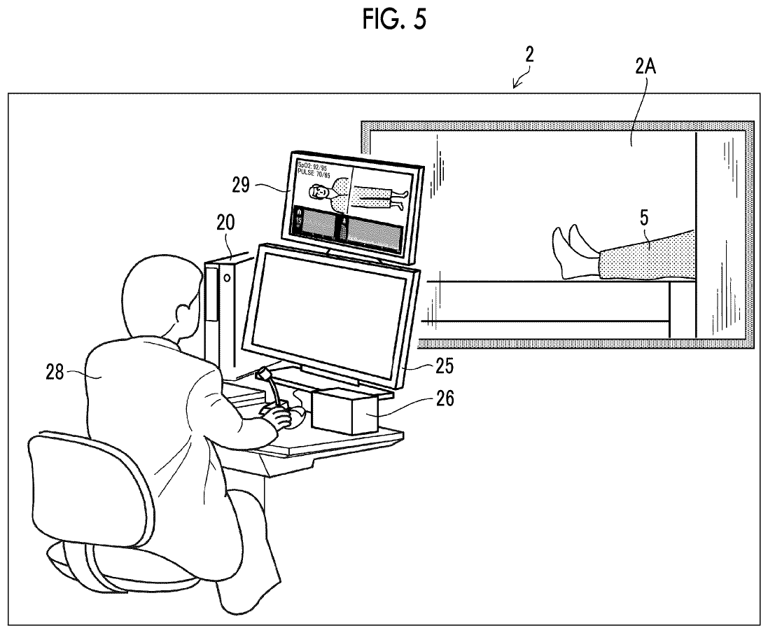

FIG. 5 is a view showing a state inside the operation room.

As shown in FIG. 5, an information processing apparatus 20 that controls the MRI apparatus 10 installed in the examination room 1, processes the NMR signal output from the MRI apparatus 10, and generates the MRI image is installed in the operation room 2.

The information processing apparatus 20 can be configured by using a computer. The computer applied to the information processing apparatus 20 may be a personal computer or may be a workstation. An operator 28 can control the operation of the MRI apparatus 10 through the information processing apparatus 20 by using an operation unit such as a keyboard and a mouse.

The information processing apparatus 20 according to the present example has a part of functions of the medical image diagnostic system and the information processing system according to the embodiment of the present invention, but the details thereof will be described below.

The information processing apparatus 20 comprises a display device (second display device) 25 that displays characters indicating the utterance content uttered by the subject during the examination, the MRI image, the vital information, and other information, and a speaker 26 that generates a voice indicating the utterance content uttered by the subject during the examination.

In FIG. 5, 2A indicates a window for the operator 28 to see the inside of the examination room 1. 29 indicates a display device that displays the video captured by the first cameras 15A and 15B.

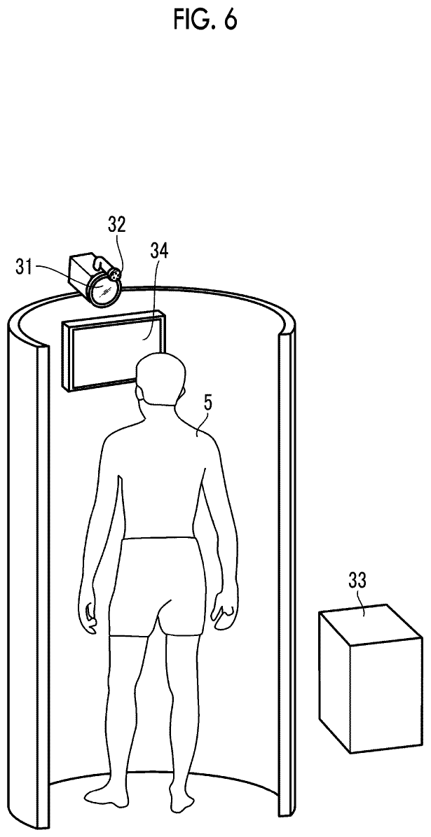

FIG. 6 is a view showing an example of equipment installed in the interview room.

As shown in FIG. 6, the interview room 3 is provided with a second camera 31, a second microphone 32, a question apparatus 33, and a display device 34.

The operator 28 causes the subject 5 to enter the interview room 3 and explains the MRI examination, performs the interview, and the like. However, in the present example, the operator 28 guides the subject 5 in front of the display device 34 and operates the question apparatus 33.

The question apparatus 33 asks a question of a predetermined format by character display by the display device 34 and a voice from a speaker (not shown).

It is preferable that the question of the predetermined format includes a question content for inducing an utterance having a possibility of being uttered by the subject during the examination using the MRI apparatus 10, as an answer, or causing the subject to read aloud the utterance.

Examples of the utterance having a possibility of being uttered by the subject during the examination include
The physical condition has deteriorated,
My ears hurt,
I feel breathless,
How long will the examination take? and the like.

Additionally, a question for identifying a person, such as a name and a date of birth, can be asked.

In addition, the question apparatus 33 may perform the explanation of the MRI examination with a video and a voice displayed on the display device 34.

The second camera 31 and the second microphone 32 each function as a detection device (second detection device) that detects response information of the subject 5 to the question to the subject 5 with a video and a voice before the examination of the subject 5 using the MRI apparatus 10 is started.

The second camera 31 can be configured by a video camera that captures the moving image (video) including the face region of the subject 5. In addition, as the second microphone 32, a microphone provided in the video camera, which is the second camera 31, can be used. As a result, the second camera 31 records and outputs the moving image (video) with a voice.

It should be noted that, in the present example, the second camera 31, the second microphone 32, the question apparatus 33, and the display device 34 are installed in the interview room 3. However, the present invention is not limited to this, and the second camera 31, the second microphone 32, the question apparatus 33, and the display device 34 may be installed in a room other than the examination room, or at a position far away from the MRI apparatus 10 in the same room as the examination room 1 (a position farther away from the MRI apparatus 10 than the first camera 15 and the first microphone 16, which are first detection devices) in a case in which the examination room 1 is sufficiently large, or conversely, only a small changing room can be provided in addition to the examination room and the operation room in a small hospital.

### Configuration Diagram of Medical Image Diagnostic System

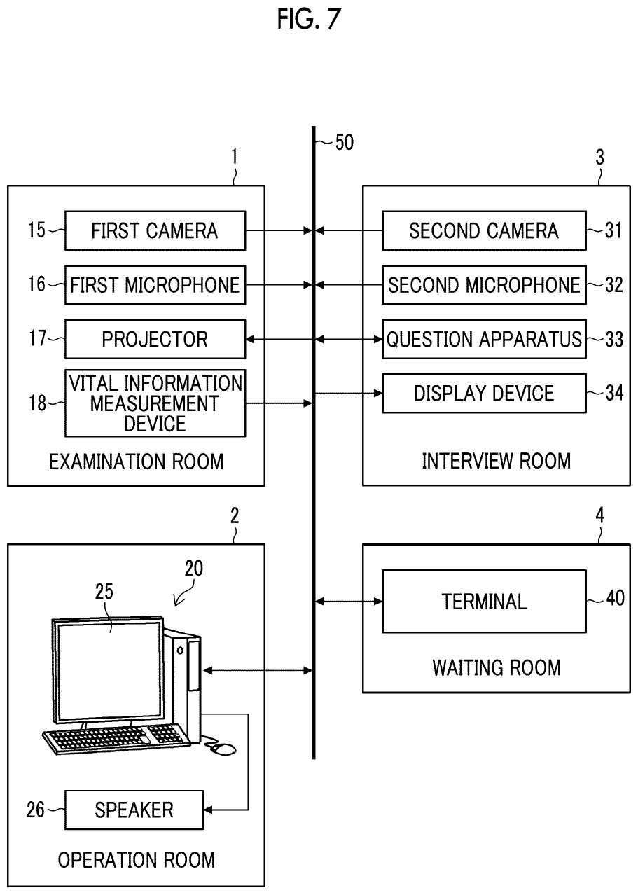

FIG. 7 is a configuration diagram of the medical image diagnostic system according to the embodiment of the present invention.

The medical image diagnostic system shown in FIG. 7 is configured such that various above-described devices installed in the examination room 1, the operation room 2, and the interview room 3 are connected to each other via a network 50, such as a local area network (LAN). In addition, a terminal 40 in the waiting room 4 can also perform communication through the network 50.

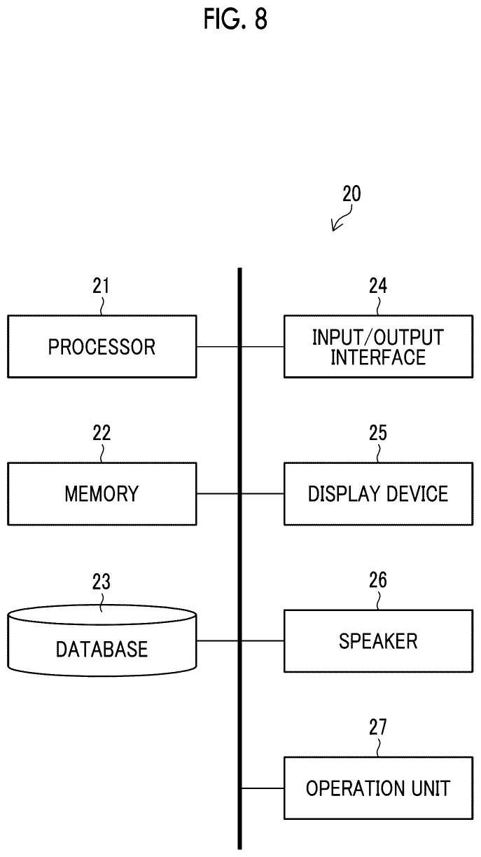

FIG. 8 is a block diagram showing an embodiment of hardware of an information processing apparatus, which is a main device constituting the medical image diagnostic system.

The information processing apparatus 20 shown in FIG. 8 is configured by the computer or the like as described above, and comprises a processor 21, a memory 22, a database 23, an input/output interface 24, the display device (second display device) 25, the speaker 26, an operation unit 27, and the like.

The processor 21 is configured by a central processing unit (CPU) and the like, and integrally controls each unit of the information processing apparatus 20 and various devices (see FIG. 7) installed in the examination room 1, the operation room 2, and the interview room 3, and executes an information processing program stored in the memory 22 to implement various functions described below.

The memory 22 is one or more memories including a flash memory, a read-only memory (ROM), a random access memory (RAM), a hard disk device, and the like. The flash memory, the ROM, and the hard disk device are non-volatile memories that store various programs including an operation system, the information processing program that causes the processor 21 to function as the information processing apparatus 20, and a machine learning model (a first machine learning model and a second machine learning model).

The RAM functions as a work area for processing by the processor 21 and temporarily stores the information processing program and the like stored in the non-volatile memory. It should be noted that a part (RAM) of the memory 22 may be incorporated in the processor 21.

The database 23 is a unit that stores and manages the response information of the video and the voice detected by the second camera 31 and the second microphone 32 of the interview room 3, and stores the response information as training data for training the first machine learning model described below.

The input/output interface 24 includes a communication unit that can be connected to the network 50, a connection unit that can be connected to an external device, and the like. For example, a universal serial bus (USB), a high-definition multimedia interface (HDMI) (HDMI is a registered trademark), or the like can be applied as the connection unit that can be connected to the external device.

The processor 21 transmits and receives necessary information to and from various devices in the examination room 1 and the interview room 3 through the input/output interface 24 and the network 50, and transmits and receives necessary information to and from the terminal 40 in the waiting room 4.

The display device 25 is used as a part of a graphical user interface (GUI) in a case in which the display device 25 receives an input from the operation unit 27, and displays the characters indicating the utterance content uttered by the subject 5 during the examination, the MRI image, the vital information, the personal information of the subject 5, and the like.

The speaker 26 generates the voice indicating the utterance content uttered by the subject 5 during the examination.

The operation unit 27 includes a mouse, a keyboard, and the like, uses a display screen of the display device 25, and functions as a part of the GUI that receives an input of the operator 28.

### Utterance Recognition Unit

An utterance recognition unit according to the present example is a function provided in the processor 21. That is, the processor 21 acquires the response information of the video and the voice detected by the second camera 31 and the second microphone 32 in the interview room 3 from the database 23, and generates subject feature information related to the voice generation of the subject 5 based on the acquired response information. The subject feature information is feature information corresponding to a habit unique to the subject 5 during the voice generation of the lip movement of the subject 5 (including the facial expression of the entire face).

In a case in which the subject 5 makes an utterance during the examination of the subject 5 using the MRI apparatus 10, the processor 21 acquires at least one of the video including the face region (lip movement of the subject 5) of the subject 5 captured by the first camera 15 or the voice of the subject 5 detected by the first microphone 16 as utterance-related information, and recognizes the utterance content of the subject 5 based on the utterance-related information and the subject feature information generated in advance.

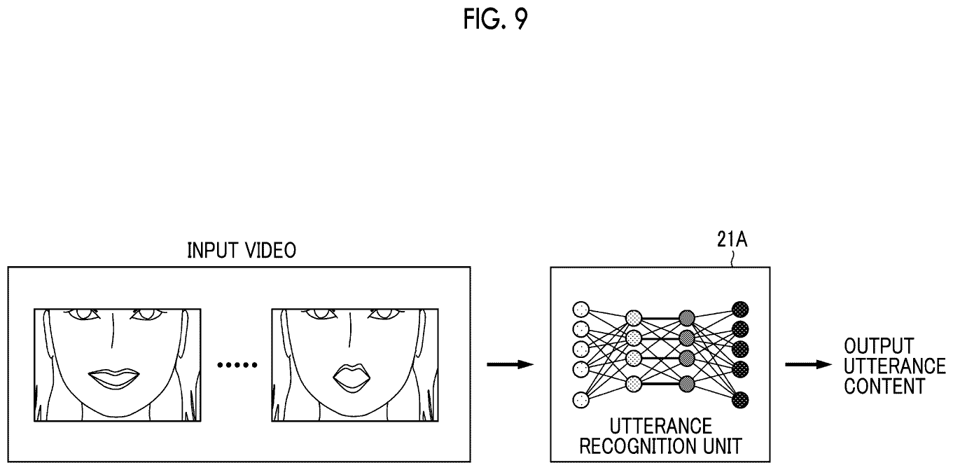

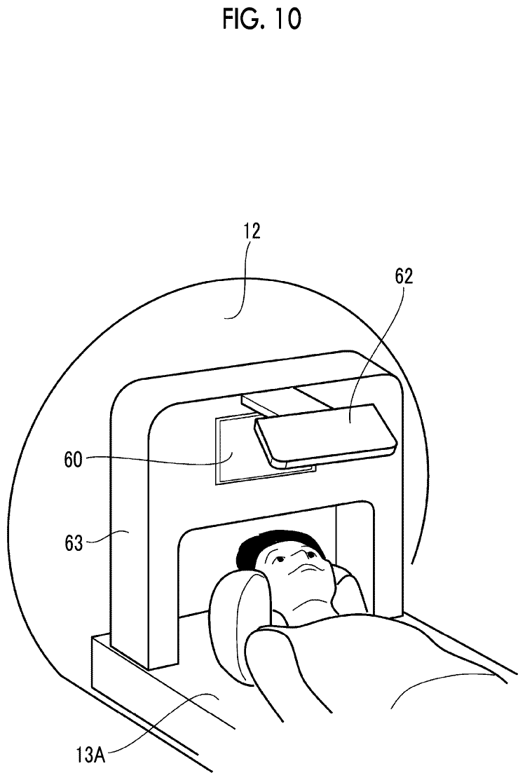

FIG. 9 is a conceptual diagram showing an embodiment of the utterance recognition unit.

As shown in FIG. 9, the processor 21 functions as an utterance recognition unit 21A.

The utterance recognition unit 21A is a lip-reading unit that reads the characters from the lip movement of the subject 5, and in the present example, uses a trained machine learning model (first machine learning model) to input the video including the face region of the subject 5 captured by the first camera 15.

The processor 21 reads out the untrained first machine learning model stored in the memory 22 or the database 23, and trains the first machine learning model.

As the first machine learning model, various learning models, such as an end-to-end deep learning network, a convolutional neural network, and a support vector machine, can be used.

A training data set used to train the first machine learning model is a plurality of pairs of the video including the face region of the subject 5 captured by the second camera 31 each time the subject 5 answers a plurality of questions in the interview room 3, and character information indicating each answer. A plurality of videos are input data in a case of training the first machine learning model, a plurality of character information are correct answer data for each corresponding video.

The character information indicating each answer can be acquired by voice recognition artificial intelligence (AI) that recognizes the characters from the voice detected by the second microphone 32. In addition, it is preferable that the characters recognized by the voice recognition AI are displayed on a monitor screen of the display device 34 in the interview room 3, and in a case in which the displayed characters are different from the answer, the subject 5 is pointed out the error and corrects the error. This is to acquire accurate correct answer data.

In a case in which a predetermined phrase or sentence is read aloud by the subject, characters indicating the predetermined phrase or sentence can be used as the correct answer data. In this case, it is preferable that the predetermined phrase or sentence is the utterance content having a possibility of being uttered by the subject during the examination using the MRI apparatus 10.

By training the first machine learning model using the training data set, it is possible to generate the trained first machine learning model dedicated to the subject. Moreover, the subject feature information includes parameters optimized in a process of training the first machine learning model based on the response information of the subject 5, and can be stored in the memory 22 or the database 23 in association with the subject.

It should be noted that, in the present example, the untrained first machine learning model is trained using the training data set acquired from the response information of the subject himself or herself, but the present invention is not limited to this, and the existing trained first machine learning model may be used, and the existing first machine learning model may be trained through transfer learning or subjected to fine tuning using the training data set acquired from the new response information of the subject himself or herself. This is effective in a case in which the number of questions is small and the number of training data sets is small.

The utterance recognition unit 21A that uses the trained first machine learning model (learning model in which the parameters optimized in the process of training the first machine learning model are set) inputs the video including the face region of the subject 5 captured by the first camera 15 during the examination of the subject 5 using the MRI apparatus 10, and in a case in which the video including the face region of the subject 5 in a case in which the subject 5 makes an utterance is input, the utterance recognition unit 21A recognizes and outputs the utterance content one character by one character.

In the present example, the utterance recognition unit 21A outputs a plurality of characters (character string) indicating the utterance content uttered by the subject 5 from the video indicating the lip movement of the subject 5 to be input, and preferably has a function of further performing natural language processing on the character string output from the utterance recognition unit 21A to determine whether or not the character string is a meaningful utterance, or performing, in a case in which there is an error as a result of performing the natural language processing on the character string, correction to a correct utterance content. Such a function can be achieved by adding existing natural language processing AI.

The utterance recognition unit may simultaneously input the video including the face region of the subject 5 captured by the first camera 15 and the voice of the subject 5 detected by the first microphone 16, and recognize the utterance content of the subject. It is preferable that the first machine learning model applied to the utterance recognition unit in this case includes a learning model that recognizes an utterance content from a video (lip movement of the subject 5) including a face region of the subject 5 and a learning model that recognizes an utterance content from a voice of the subject 5, and uses a learning model in which both learning models are trained to show the same utterance content. As this type of first machine learning model, the end-to-end deep learning network is suitable.

In addition, by recognizing the utterance content from the lip movement and the voice of the subject 5, it is possible to recognize the utterance content with higher accuracy. It should be noted that, in a case in which the voice detected by the first microphone 16 is input to the first learning model, it is preferable to reduce the noise generated by the MRI apparatus 10 in advance by a filter or the like.

In a case in which the subject 5 utters a voice during the examination using the MRI apparatus 10 as described above and the processor 21 recognizes the utterance content of the subject 5, the processor 21 projects the video (characters) indicating the recognized utterance content from the projector 17 onto the screen 17A, and displays the video (characters) on the screen 17A (see FIG. 3).

The subject 5 can visually recognize the utterance content uttered by the subject 5 himself or herself and projected onto the screen 17A. As a result, the subject 5 can confirm that his or her utterance content is transmitted to the external operator or the like, and can obtain a sense of security.

The processor 21 may project a video for relaxing the subject 5 (for example, a video of nature) or a video of an attendant to the screen 17A from the projector 17. In this case, in a case in which the subject 5 utters a voice, the processor 21 projects the utterance content onto the screen 17A instead of the video, or superimposes the utterance content on the video and projects the superimposed video to the screen 17A.

In addition, in a case in which the processor 21 recognizes the utterance content of the subject 5, the processor 21 displays characters indicating the recognized utterance content on the display device 25 of the operation room 2, and generates a voice indicating the utterance content from the speaker 26 in the same manner (see FIG. 5). Accordingly, the operator 28 can confirm the utterance content uttered by the subject 5 during the examination.

Further, in a case in which the processor 21 recognizes the utterance content of the subject 5, the processor 21 can display the characters indicating the recognized utterance content on the terminal 40 of the waiting room 4. As a result, the attendant of the subject 5 can confirm the utterance content uttered by the subject 5 during the examination by the terminal 40 of the waiting room 4.

### Another Embodiment of First Display Device

FIG. 10 is a diagram showing another embodiment of the first display device that displays information in an aspect visible to the subject during the examination using the MRI apparatus.

A first display device shown in FIG. 10 includes a projector (not shown), a rear projection screen 60 on which the characters indicating the utterance content of the subject 5 are projected from the projector, and a reflecting mirror 62 for allowing the subject 5 lying face up on the top plate 13A to visually recognize the rear projection screen 60.

The rear projection screen 60 and the reflecting mirror 62 are provided in a support member 63 attached to the top plate 13A.

In a case in which the characters indicating the utterance content of the subject 5 are projected onto the rear projection screen 60 from the projector, the subject 5 can visually recognize the characters indicating the utterance content of the subject 5 projected onto the rear projection screen 60 via the reflecting mirror 62. The first camera that images the face region of the subject 5 can be attached to an outer peripheral portion of the reflecting mirror 62.

The other embodiment of the first display device that displays information in an aspect visible to the subject during the examination using the MRI apparatus is not limited to the embodiment shown in FIG. 10, and a head-up display that displays a virtual image (air image), a head-mounted display that is mounted on the head of the subject, a liquid crystal display, or an organic electro-luminescence (EL) display can be used.

### Operation Method of Medical Image Diagnostic System

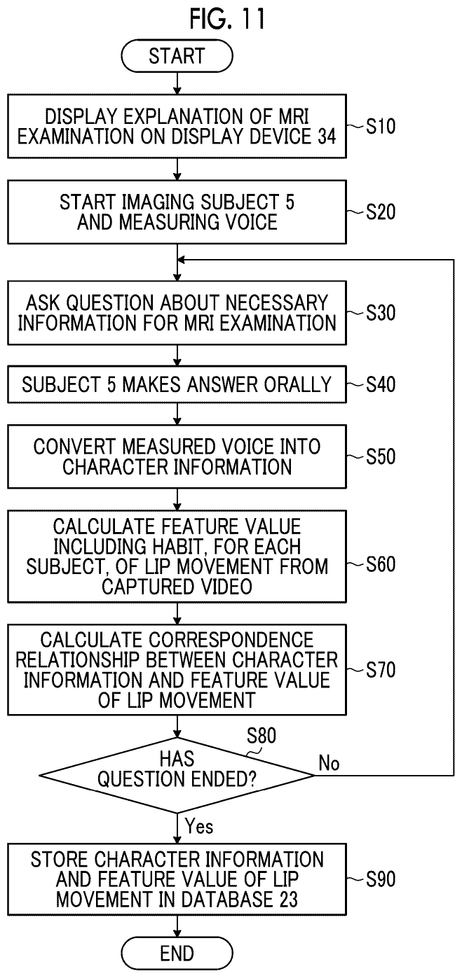

FIG. 11 is a flowchart showing an embodiment of the operation method of the medical image diagnostic system that is operated in a case in which the subject is in the interview room.

The processing of each step of the operation method of the medical image diagnostic system shown in FIG. 11 corresponds to processing performed by the processor 21 of the information processing apparatus 20 shown in FIG. 8.

In FIG. 11, the processor 21 displays the explanation of the MRI examination on the display device 34 installed in the interview room 3 (step S10). Here, a procedure of the MRI examination, cautions during the MRI examination, and the like are displayed.

Then, the processor 21 operates the second camera 31 and the second microphone 32 to start detecting the response information of the subject 5 to the question to the subject 5 with a video and a voice (step S20).

The processor 21 asks the question of the predetermined format (for example, a question about information necessary for the MRI examination) by character display via the display device 34 and a voice from a speaker (not shown) (step S30).

The subject answers the question in step S30 orally (step S40).

The processor 21 converts the voice of the subject 5 answered orally into the character information, the voice being detected by the second microphone 32 (step S50). The conversion of the voice into the character information can be performed by using the voice recognition AI.

In addition, the processor 21 calculates a feature value including the habit, for each subject, of the lip movement of the subject 5 captured by the second camera 31 in the video including the face region of the subject 5 who answers orally (step S60), and calculates a correspondence relationship between the character information converted in step S50 and the feature value of the lip movement (step S70).

Then, the processor 21 determines whether or not the question has ended (step S80), and in a case in which it is determined that the question has not ended (in a case of “No”), the processor 21 proceeds to step S30 and repeats the processing from step S30 to step S80 for the next question.

In a case in which the processor 21 determines that all the questions has ended (in a case of “Yes”), the processor 21 stores the character information and the feature value of the lip movement, which are calculated in step S70, in the database 23 (step S90).

The character information and the feature value of the lip movement stored in the database 23 are used for the lip reading during the examination.

In the present example, by repeating the processing from step S30 to step S80, the first machine learning model is trained, and the optimized parameters of the trained first machine learning model are stored in the database 23 as the subject feature information unique to the subject 5.

It should be noted that the processor 21 preferably displays the character information converted in step S50 on the display device 34, confirms whether or not the converted character information matches the content answered orally by the subject 5, and in a case in which the converted character information does not match the content answered orally by the subject 5, orally points out a difference and corrects the character information.

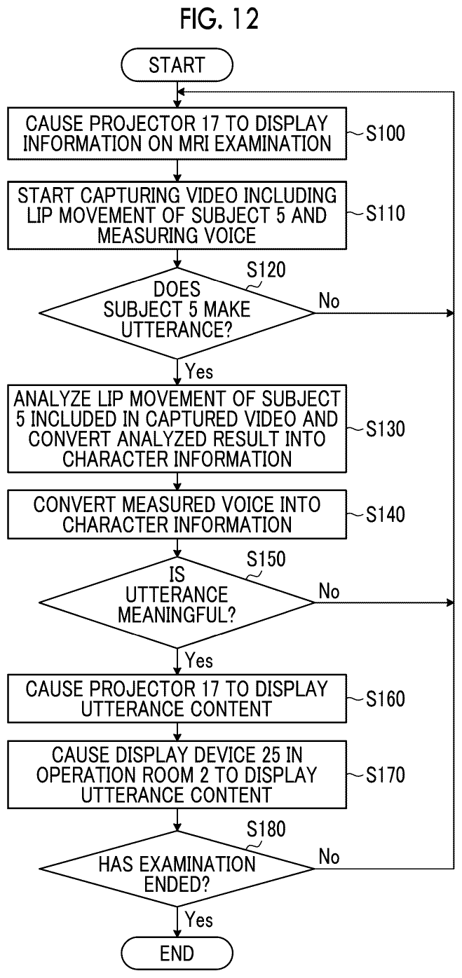

FIG. 12 is a flowchart showing a first embodiment of the operation method of the medical image diagnostic system during the MRI examination.

The processing of each step of the operation method of the medical image diagnostic system shown in FIG. 12 corresponds to processing performed by the processor 21 of the information processing apparatus 20 shown in FIG. 8.

In FIG. 12, the processor 21 displays information on the MRI examination via the projector 17 (step S100). The subject 5 during the examination can visually recognize the information displayed by the projector 17. It should be noted that, in step S100, information other than the information on the MRI examination can be displayed. For example, the video of the attendant or the video of the nature can be displayed, and thus the subject 5 can be relaxed.

Then, the processor 21 operates the first camera 15 and the first microphone 16 to start capturing the video including the lip movement of the subject 5 during the examination and measuring the voice uttered by the subject 5 (step S110).

The processor 21 determines whether or not the subject 5 makes an utterance (step S120). Whether or not the subject 5 makes an utterance can be determined by detecting whether or not the subject 5 has a lip movement from the video captured by the first camera 15 and/or by detecting whether or not the subject 5 utters a voice from the first microphone 16.

In a case in which the processor 21 determines that the subject 5 does not make an utterance (in a case of “No”), the processor 21 proceeds to step S100 and repeats the processing from step S100 to step S120.

On the other hand, in a case in which the processor 21 determines that the subject 5 makes an utterance (in a case of “Yes”), the processing proceeds to step S130.

In step S130, the lip movement of the subject 5 included in the video captured by the first camera 15 is analyzed (lip reading) and converted into the character information. In the present example, the parameters of the trained first machine learning model, which are acquired in advance for the subject 5 and stored in the database 23, are read out and set in the first machine learning model, and the video including the lip movement of the subject 5 is input to the utterance recognition unit using the first machine learning model, thereby recognizing the character information corresponding to the lip movement. It should be noted that the character information may be recognized by collectively using the information on the entire face in addition to the lip movement.

In addition, the processor 21 converts the voice uttered by the subject 5 detected by the first microphone 16 into the character information (step S140). The processor 21 can use the voice recognition AI to convert the voice into the character information.

The processor 21 can improve the accuracy of the converted character information by adding the voice-recognized character information in addition to the character information recognized in response to the lip movement and recognizing the character information using both of the character information.

Then, the processor 21 determines whether or not the converted character information (character string) is a meaningful utterance (step S150). Whether or not the utterance is meaningful can be determined by natural language processing for the character string. In a case in which it is determined that the utterance is not meaningful (in a case of “No”), the processing proceeds to step S100.

On the other hand, in a case in which it is determined that the utterance is meaningful (in a case of “Yes”), the processor 21 causes the projector 17 to display the characters indicating the utterance content (step S160).

As a result, the subject 5 during the examination can visually recognize the characters indicating the utterance content uttered by the subject 5 himself or herself. As a result, the subject 5 can confirm that his or her utterance content is transmitted to the external operator or the like, and can obtain a sense of security.

In addition, in a case in which the processor 21 determines that the utterance is meaningful (in a case of “Yes”), the processor 21 also causes the display device 25 (second display device) of the operation room 2 to display the characters indicating the utterance content (step S170). Accordingly, the operator 28 in the operation room 2 can confirm the utterance content uttered by the subject 5 during the examination. It should be noted that a notification device that notifies the operator 28 in the operation room 2 of the utterance content can be configured by at least one of the display device 25 that displays the characters indicating the utterance content or the speaker 26 that generates the voice indicating the utterance content.

Then, the processor 21 determines whether or not the MRI examination has ended (step S180), and in a case in which it is determined that the MRI examination has not ended (in a case of “No”), the processor 21 proceeds to step S100 and repeats the processing from step S100 to step S180.

On the other hand, in a case in which it is determined that the MRI examination has ended (in a case of “Yes”), the processor 21 ends the present processing.

It should be noted that the processor 21 may also display the characters indicating the utterance content uttered by the subject 5 on the terminal 40 in the waiting room 4.

In the present example, only the meaningful utterance is displayed, but the meaningless utterance may be displayed. This is because it is considered that the subject 5 feels anxious in a case in which nothing is displayed even though the subject 5 utters a voice during the examination.

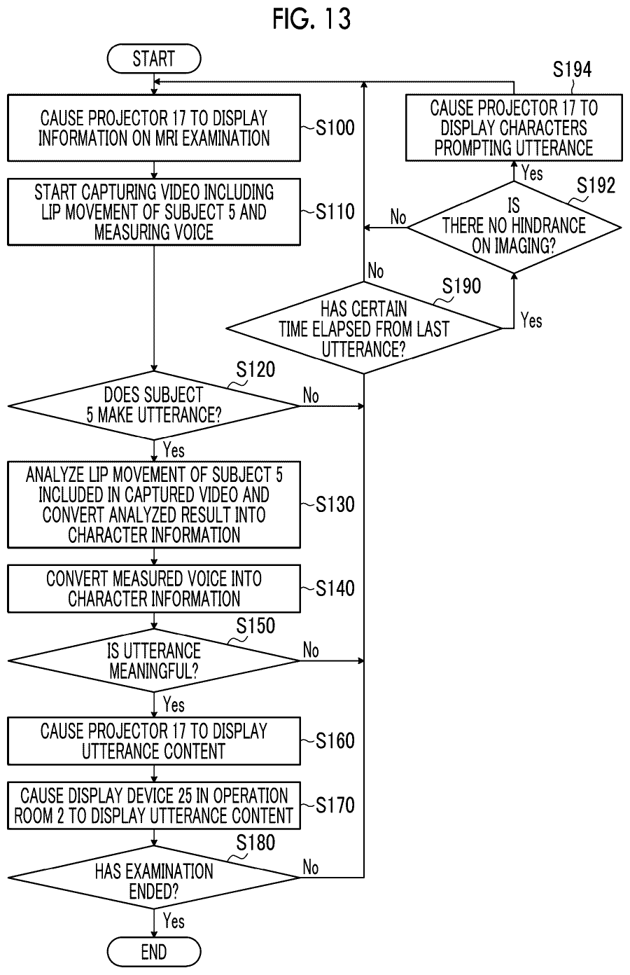

FIG. 13 is a flowchart showing a second embodiment of the operation method of the medical image diagnostic system during the MRI examination.

It should be noted that, in FIG. 13, the same parts as parts in the flowchart shown in FIG. 12 are denoted by the same step number, and detailed description thereof will be omitted.

The operation method of the medical image diagnostic system according to the second embodiment shown in FIG. 13 is different from the first embodiment in that pieces of processing of steps S190, S192, and S194 are added.

In FIG. 13, on the flow line returning to step S100, the processor 21 determines whether or not a certain time has elapsed from the last utterance (step S190). A certain time can be an appropriate time that can be determined as the silence after the utterance.

In a case in which it is determined that a certain time has elapsed (“Yes”), the processor 21 further determines whether or not the utterance of the subject 5 hinders the MRI imaging (MRI examination) (step S192). Whether or not the utterance of the subject 5 hinders the MRI imaging can be determined by the examination part set in advance. That is, it can be determined that the body movement (movement of the head) due to the utterance of the subject 5 does not hinder the examination of the part other than the head. Therefore, the processor 21 can determine that there is no hindrance to the MRI imaging in a case of the examination of the part other than the head.

In a case in which it is determined that a certain time has elapsed from the last utterance and there is no hindrance on the MRI imaging, the processor 21 displays characters prompting the subject 5 to utter a voice on the projector 17 (step S194).

As a result, the communication between the subject 5 and the operator 28 can be performed, and the anxiety of the subject 5 can be resolved.

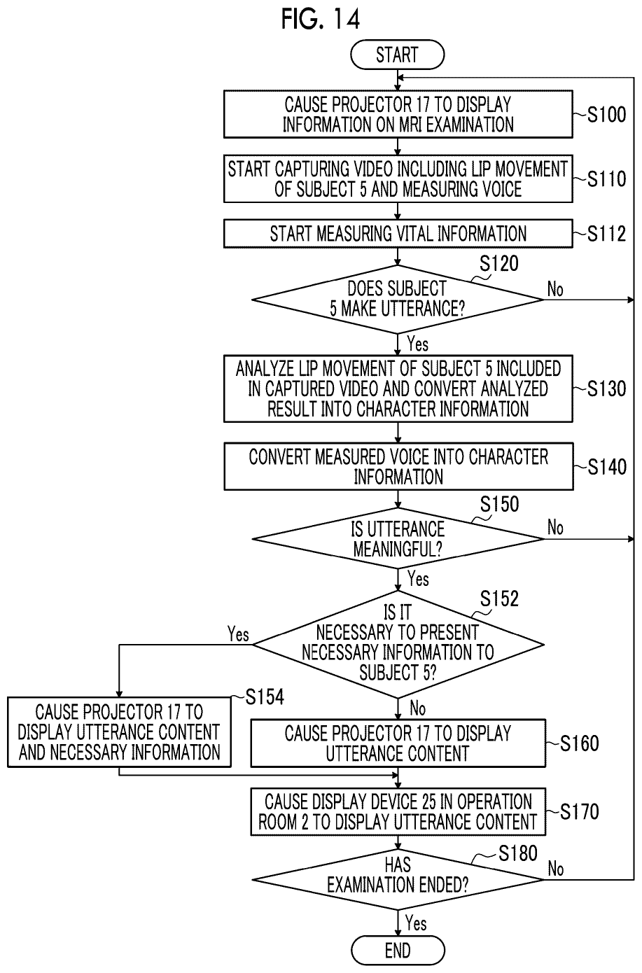

FIG. 14 is a flowchart showing a third embodiment of the operation method of the medical image diagnostic system during the MRI examination.

It should be noted that, in FIG. 14, the same parts as parts in the flowchart shown in FIG. 12 are denoted by the same step number, and detailed description thereof will be omitted.

The operation method of the medical image diagnostic system according to the third embodiment shown in FIG. 14 is different from the first embodiment in that pieces of processing of steps S112, S152, and S154 are added.

In FIG. 14, in step S112, measuring the vital information of the subject 5 via the vital information measurement device 18 (see FIG. 3) is started. Here, the vital information measurement device 18 measures one or more of the heart rate, the blood pressure, the respiratory rate, the body temperature, the electrocardiogram, or the blood oxygen saturation concentration of the subject 5 as the vital information of the subject 5.

In step S152, it is determined whether or not it is necessary to present information (a reply to the utterance content) necessary for the subject 5 from the utterance content uttered by the subject 5 and the vital information of the subject 5 measured in step S112, and in a case in which it is determined that the reply is necessary, a reply sentence to the utterance content is further created.

For example, in a case in which a significant increase in the heart rate or the respiratory rate is detected, it is considered that the user feels anxious or is nervous because he or she cannot endure a long-term examination or a closed space. On the other hand, in a case in which the subject 5 feels anxious, it is preferable to present information that relieves the anxiety of the subject 5 as the information that requires the reply from the subject 5. A reply sentence such as “Are you feeling okay?”, “The examination will end in a little while, but are you okay?”, or the like is created. In addition, the video of the face of the operator 28 or the face of the attendant can be used as the necessary information.

In a case in which the body temperature of the examination part is increased and the subject 5 complains of being “hot”, a reply sentence such as “The temperature is rising in the examination, but can you endure it?” is created.

In a case in which it is determined that it is necessary to provide the necessary information to the subject 5 (in a case of “Yes”), the processor 21 displays the characters indicating the utterance content of the subject 5 via the projector 17 and displays the created necessary information (reply sentence) (step S154).

On the other hand, in a case in which the processor 21 determines that it is not necessary to provide the necessary information to the subject 5 (in a case of “No”), the processor 21 proceeds to step S160 and displays only the characters indicating the utterance content of the subject 5 via the projector 17.

### Others

In the present embodiment, the first machine learning model dedicated to the subject is generated, the video including the face region of the subject during the MRI examination is input to the utterance recognition unit using the first machine learning model to analyze the lip movement (read lips) in a case in which the subject generates the voice, and the character information indicating the utterance content is acquired as the recognition result. However, for example, in a case in which the utterance content recognized by the first machine learning model is not a meaningful content or in a case in which a certainty degree of the utterance content is less than a threshold value, another machine learning model (second machine learning model) may be used or used in combination.

As the second machine learning model in this case, a learning model that has been trained through machine learning in advance based on a large number of training data sets consisting of the utterance-related information related to the utterances of a plurality of people, and that is stored in advance in the memory 22 or the database 23 can be used.

In the present embodiment, for example, the hardware structure of the processing unit that executes various types of processing, such as a CPU, include the following various processors. The various processors include, for example, a central processing unit (CPU) which is a general-purpose processor executing software (program) to function as various processing units, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform specific processing.

One processing unit may be configured by one of these various processors or by two or more processors of the same type or different types (for example, a plurality of FPGAs or a combination of a CPU and an FPGA). Moreover, a plurality of processing units may be configured by one processor. A first example of the configuration in which a plurality of processing units are configured by one processor is a form in which one processor is configured by a combination of one or more CPUs and software and functions as a plurality of processing units, as represented by a client computer or a server computer. A second example of the configuration is a form in which a processor that implements the functions of the entire system including a plurality of processing units using one integrated circuit (IC) chip is used, as represented by a system on chip (SoC). As described above, various processing units are configured by using one or more of the various processors as the hardware structure.

More specifically, the hardware structure of these various processors is an electric circuit (circuitry) obtained by combining circuit elements such as semiconductor elements.

Further, it is needless to say that the present invention is not limited to the above-described embodiments and various modifications can be made without departing from the gist of the present invention.

### EXPLANATION OF REFERENCES

1: examination room
2: operation room
3: interview room
4: waiting room
10: MRI apparatus
11: gantry
12: bore
13: bed
13A: top plate

What is claimed is:

1. A medical image diagnostic system comprising:
an image diagnostic apparatus that acquires a medical image;
a first detection device that detects, with at least one of a video or a voice, utterance-related information related to an utterance of a subject during an examination of the subject using the image diagnostic apparatus;
a second detection device that detects, with at least one of a video or a voice, response information of the subject to a question to the subject before the examination of the subject using the image diagnostic apparatus is started;
a first display device that displays information in an aspect visible to the subject during the examination using the image diagnostic apparatus;
a processor; and
a memory that stores a program to be executed by the processor,
wherein the processor
generates subject feature information related to voice generation of the subject based on the response information detected by the second detection device, and
recognizes an utterance content of the subject based on the utterance-related information detected by the first detection device and the subject feature information, to cause the first display device to display the recognized utterance content.

2. The medical image diagnostic system according to claim 1,
wherein the first detection device is provided in a vicinity of the image diagnostic apparatus or the image diagnostic apparatus, and includes at least one of a first camera that captures a video including at least a lip part of a face region of the subject during the examination using the image diagnostic apparatus, or a first microphone that detects a voice uttered by the subject.

3. The medical image diagnostic system according to claim 2,
wherein the utterance-related information is a lip movement of the subject acquired from the video captured by the first camera.

4. The medical image diagnostic system according to claim 1,
wherein the second detection device is provided at a position farther away from the image diagnostic apparatus than the first detection device, and includes at least one of a second camera that captures a video including a face region of the subject, or a second microphone that detects a voice uttered by the subject.

5. The medical image diagnostic system according to claim 4,
wherein the response information is a lip movement of the subject acquired from the video captured by the second camera.

6. The medical image diagnostic system according to claim 1, further comprising:
a question apparatus that asks a question to the subject,
wherein the question apparatus asks a question of a predetermined format with at least one of a voice or characters on a monitor screen.

7. The medical image diagnostic system according to claim 6,
wherein the question of the predetermined format includes a question content for inducing an utterance having a possibility of being uttered by the subject during the examination, as an answer, or causing the subject to read aloud the utterance.

8. The medical image diagnostic system according to claim 1,
wherein the processor
trains a first machine learning model dedicated to the subject based on the response information detected by the second detection device, and
inputs the utterance-related information detected by the first detection device to the trained first machine learning model, to acquire the utterance content recognized by the first machine learning model.

9. The medical image diagnostic system according to claim 8,
wherein the subject feature information includes parameters optimized in a process of training the first machine learning model based on response information of the subject.

10. The medical image diagnostic system according to claim 8,
wherein a second machine learning model that has been trained through machine learning in advance based on a training data set consisting of utterance-related information related to utterances of a plurality of people is provided, and
the processor inputs the utterance-related information detected by the second detection device to the second machine learning model, to acquire the utterance content recognized by the second machine learning model in a case in which the utterance content recognized by the first machine learning model is not a meaningful content or a certainty degree of the utterance content is less than a threshold value.

11. The medical image diagnostic system according to claim 1, further comprising:
a notification device that notifies an operator in an operation room of the image diagnostic apparatus of the utterance content,
wherein the processor outputs the utterance content to the notification device.

12. The medical image diagnostic system according to claim 11,
wherein the notification device is at least one of a second display device that displays characters indicating the utterance content or a speaker that generates a voice indicating the utterance content.

13. The medical image diagnostic system according to claim 3,
wherein the processor
determines whether or not the lip movement of the subject hinders the examination of the subject using the image diagnostic apparatus in a case in which next utterance-related information is not detected for a certain time or longer after the first detection device detects the utterance-related information, and causes the first display device to display characters prompting the subject to make an utterance in a case in which it is determined that the lip movement of the subject does not hinder the examination of the subject using the image diagnostic apparatus.

14. The medical image diagnostic system according to claim 1, further comprising:
a vital information measurement device that measures vital information of the subject during the examination of the subject using the image diagnostic apparatus,
wherein the processor determines whether or not a reply to the utterance content is necessary, based on the utterance content and the measured vital information, and creates a reply sentence corresponding to the utterance content to cause the first display device to display the reply sentence in a case in which it is determined that the reply is necessary.

15. The medical image diagnostic system according to claim 14,
wherein the vital information measurement device measures one or more of a heart rate, a blood pressure, a respiratory rate, a body temperature, an electrocardiogram, or a blood oxygen saturation concentration of the subject.

16. The medical image diagnostic system according to claim 1,
wherein the first display device is a projector that performs projection onto a screen visible to the subject, a head-up display, a head-mounted display, a liquid crystal display, or an organic EL display.

17. The medical image diagnostic system according to claim 1,
wherein the image diagnostic apparatus includes a magnetic resonance imaging apparatus, an X-ray CT apparatus, a PET apparatus, a radiation therapy apparatus, or a particle beam therapy apparatus.

18. An operation method of a medical image diagnostic system including a first detection device that detects, with at least one of a video or a voice, utterance-related information related to an utterance of a subject during an examination of the subject using an image diagnostic apparatus, a second detection device that detects, with at least one of a video or a voice, response information of the subject to a question to the subject before the examination of the subject using the image diagnostic apparatus is started, a first display device that displays information in an aspect visible to the subject during the examination using the image diagnostic apparatus, a processor, and a memory that stores a program to be executed by the processor, the operation method comprising:
a step of generating, via the processor, subject feature information related to voice generation of the subject based on the response information detected by the second detection device;
a step of recognizing, via the processor, an utterance content of the subject based on the utterance-related information detected by the first detection device and the subject feature information; and
a step of causing, via the processor, the first display device to display the recognized utterance content.

19. The operation method of a medical image diagnostic system according to claim 18, further comprising:
a step of training, via the processor, a first machine learning model dedicated to the subject based on the response information detected by the second detection device; and
a step of inputting, via the processor, the utterance-related information detected by the first detection device to the trained first machine learning model, to acquire the utterance content recognized by the first machine learning model.

20. An information processing system comprising:
a first detection device that detects, with at least one of a video or a voice, utterance-related information related to an utterance of a subject during an examination of the subject using an image diagnostic apparatus;
a second detection device that detects, with at least one of a video or a voice, response information of the subject to a question to the subject before the examination of the subject using the image diagnostic apparatus is started;
a first display device that displays information in an aspect visible to the subject during the examination using the image diagnostic apparatus;
a processor; and
a memory that stores a program to be executed by the processor,
wherein the processor
generates subject feature information related to voice generation of the subject based on the response information detected by the second detection device, and
recognizes an utterance content of the subject based on the utterance-related information detected by the first detection device and the subject feature information, to cause the first display device to display the recognized utterance content.
